(19) United States
(12) Patent Application Publication
(10) Pub. No.: US 2010/0330675 A1
(43) Pub. Date: Dec. 30, 2010

(54) INSULATING POLYNUCLEOTIDES DERIVED FROM ELEMENT D4Z4 AND THEIR USES IN TRANSGENESIS

(75) Inventors: Frederique Magdinier, Vaulx En Velin (FR); Eric Gilson, Lyon (FR); Alexandre Ottaviani, Lyon (FR); Sylvie Gervier Rival, Portes Les Valence (FR)

Correspondence Address: Pepper Hamilton LLP, 400 Berwyn Park, 899 Cassatt Road, Berwyn, PA, 19312-1183, US

(73) Assignees: ENS - ECOLE NORMALE SUPERIEURE DE LYON, Lyon (FR); UNIVERSITE CLAUDE BERNARD LYON 1, Villeurbanne (FR); INSTITUT NATIONAL DE LA RECHERCHE AGRONOMIQUE, Paris (FR)

(21) Appl. No.: 12/671,586

(22) PCT Filed: Jul. 30, 2008
(86) PCT No.: PCT/EP2008/060004

§ 371 Date: Sep. 10, 2010

(30) Foreign Application Priority Data
Aug. 2, 2007 (FR) 0756890

(51) Int. Cl. C12N 15/79; C07H 21/04; C12N 5/071

(52) U.S. Cl. 435/455; 536/23.1; 435/320.1; 435/325

(57) ABSTRACT

Polynucleotides with insulating properties allowing protection of the expression of a transgene from adjacent cis elements in higher eukaryotic cells. In particular, these polynucleotides can be used for transgenesis, gene therapy and the production of recombinant proteins.

This invention relates to polynucleotides with insulating properties allowing protection of the expression of a transgene from adjacent cis elements in higher eukaryotic cells. The invention also relates to the use of these polynucleotides for transgenesis, gene therapy and the production of recombinant proteins by protecting the expression of transgenes and endogenous sequences from the effect of transgenes.

The normal functioning of a cell at a particular point in time and in a given tissue results from a combination of finely tuned activation and inactivation mechanisms which allow the cell to display a particular phenotype. The specificity and mode of expression of each gene is determined by the DNA sequence controlled by structural regulations in the chromatin fibre. The degree of chromatin compaction reflects the transcriptional activity of genes found in the cell. More precisely, active genes are generally contained in regions that are not very compact (euchromatin state) which are easily accessed by the protein mechanism leading to transcription. On the other hand, inert DNA regions or those not containing active genes are usually condensed (heterochromatin state). The formation of heterochromatin in a genome region can trigger the repression of large areas of DNA (silencing). When a transgene is incorporated close to the heterochromatin region, it undergoes loss of its expression over the course of time, called “position effect”. This position-effect variegation (PEV) of a heterochromatin region on a gene can be exerted even when the distance from the gene is considerable. Equally, in different organisms, telomers located at the extremity of the chromosomes can have a repression effect on the expression of genes located nearby. This is called the telomeric position effect or TPE.

In parallel to the respective role of these chromatin states, there also exist within the genome elements which control the expression of genes, by inhibition or activation of the transcription of these genes. These cis-regulating elements are not necessarily located close to the target gene and can sometimes be located at a considerable distance from their target. It has been shown that the formation of loops is probably involved in these mechanisms.

All these internal regulations of the genome represent a problem in terms of incorporating exogenous DNA into the genome. Certain methods make it possible to target the exogenous DNA incorporation sites. In other applications, when incorporation of the exogenous gene is carried out randomly, the degree of gene expression is dependent on the structure of chromatin and the presence of cis-regulating elements which can affect transcription of this gene. The degree of gene expression is therefore unpredictable.

A solution to this problem is the use of so-called “insulating” or “isolating” elements which protect the gene from neighbouring regulating elements. These insulators are defined by two properties which can be combined or separate. They can block the interaction between a transcription regulator and promoter when they are placed between the two elements and/or limit the propagation of condensed chromatin zones towards neighbouring regions and therefore limit its positive or negative effects.

Various insulators have been described and characterised in eukaryotes. In higher eukaryotes, the best known insulator is that of the beta-globin locus in chicken (Chung J H, Whiteley M, Felsenfeld G. (1993). Cell. 74(3):505-14). This locus contains several genes regulated by a common element called LCR which controls the expression of various isoforms in the course of embryogenesis and the differentiation of the haematopoietic line. This locus is bordered on the one hand by a set of genes coding for the olfactory receptors and, on the other hand, by a condensed chromatin region. A 1.2 kb element located upstream of this locus has been characterised for its insulating properties. The insulating function is contained in a sequence of only 242 base pairs containing 5 putative protein binding sites (Chung J H, Whiteley M, Felsenfeld G. (1993). Cell. 74(3):505-14; Bell A C, West A G, Felsenfeld G. (1999). Cell. 98(3):387-96). Site n^{o} 2 binds CTCF protein which is the only protein with insulating activity described to date in vertebrates. This protein binds a 42 base pair DNA motif and makes it possible to block the transcription regulators but does not protect against position-effect variegation. This second effect is mediated by other partially characterised sequences. This insulator has been described in several patent applications (WO94/023046; WO96/004390; WO01/002553) and is used in various types of applications (Malik P, Arumugam P I, Yee J K, Puthenveetil G. (2005). Ann N Y Acad Sci. 1054:238-49; Puthenveetil G, Scholes J, Carbonell D, Qureshi N, Xia P, Zeng L, Li S, Yu Y, Hiti A L, Yee J K, Malik P. (2004). Blood. 104(12):3445-53).

Nevertheless, this insulator is sometimes not powerful enough to limit variegation of transgene expression (van Meerten T, Claessen M J, Hagenbeek A, Ebeling S B. (2006). Gene Ther. 13(9):789-97; Martin-Duque P, Jezzard S, Kaftansis L, Vassaux G. (2004). Hum Gene Ther. 15(10):995-1002). In fact, the use of two copies of this element is generally found to be necessary, suggesting that in certain contexts the insulation mechanisms mediated by this element do not allow the chromatin effect to be counteracted.

Other elements with total or partial insulating activity identified in higher eukaryotes have been described in the literature (Majem M, Cascallo M, Bayo-Puxan N, Mesia R, Germa J R, Alemany R. (2006). Cancer Gene Ther. 13(7):696-705; Kalos M, Fournier R E. (1995). Mol Cell Biol. 15(1):198-207) or have been the subject of patent applications (WO 04/065581).

The objective of this invention is therefore to propose new polynucleotides with strong insulating properties. The polynucleotides of this invention are derived from a sequence called D4Z4.

D4Z4 is a human repetitive sequence whose insulating role has never been described to date. A D4Z4 sequence was registered in Genbank under access number D38024. Deletion of this sequence at the telomeric end of the long arm of chromosome 4 (locus 4q35) is involved in facioscapulo-humeral muscular dystrophy (FSHD). Element D4Z4 is found in varying numbers of copies in locus 4q35. The number of sequences ranges from 11 to more than 100 in normal individuals and is generally below 10 in individuals with the disease. Identical sequences to D4Z4 are found in other areas of the genome, close to the telomeres (10q26) of the heterochromatin region on chromosome 1 and the acrocentric chromosomes. The precise role of D4Z4 at these different loci is not known but they are not associated with any pathology. The subtelomeric changes observed between homologous sequences at 4q35 and 10p26, without any association with clinical symptoms of FSHD, indicates that D4Z4 does not code for the gene responsible for this disease and that the number rather than the D4Z4 sequence itself is important in the aetiology of this dystrophy. It is therefore important to point out that isolated D4Z4 is not the cause of any disease and that only the particular chromatin complex of locus 4q35 induces pathological manifestations. Thus, changing the organisation of chromatin linked to the deletion of a certain number of D4Z4 elements at the 4q35locus may modify the expression of various genes close to this element or at a considerable distance from chromosome 4 (van der Maarel S M, Frants R R. (2005). Am J Hum Genet. 76(3):375-86).

Element D4Z4 has been fully sequenced without its function being identified. It has characteristics that are similar to human repeated regions. Moreover, this sequence contains a potential coding region (ORF, Open Reading Frame) for a double homeodomain protein (Dux4). However, no transcripts have been detected to date. This sequence also contains a 27 base pair DNA sequence which potentially binds to a repressor protein complex (Gabellini D, Green M R, Tupler R. (2002). Cell. 110(3):339-48; WO/2005/037231) (FIG. 1). This element presents no homology with the insulating element sequence of the beta-globin locus in chicken nor any known insulator in any other species.

Tam et al. (Journal of Cell Biology, 167::2:269-279, 2004) explains that D4Z4 repeats constitute a family of subtelomeric repeats present on human chromosomes but that their potential function is not known. This document discusses the possibility that D4Z4 could function as a silencer element or as an insulator. Further work is needed to explore the role and function of D4Z4.

Silvère et al. (Am. J. Hum. Genet., 76:375-386, 2005) is a review article on the D4Z4 repeat mediated pathogenesis of FSHD. Different models are discussed in which D4Z4 may operate as a silencer or as an insulator separating distal heterochromatin from proximal genes.

Parnell et al. (Encyclopedia of Life Sciences, 1-5, 2006) discuss position effect variegation in human genetic disease. According to this document D4Z4 could provide a buffer from telomeric silencing or could contribute to silencing of 4q genes.

Petrov et al. (PNAS, 103:18:6982-6987, 2006) investigates chromatin loop domain organization within the 4q35 locus in FSHD. This document is silent with respect to any insulating properties of D4Z4.

Ottavani et al. (Neuromuscular disorders, 16:644-726, 2006) discuss the role of the tandemly repeated D4Z4 element in FSHD. Ottavani et al. propose the hypothesis that D4Z4 may protect from position effect variegation and telomere position effect. However, no data is shown and smaller fragments derived from D4Z4 are not described.

Levelle C. and Sigal A. (Chromosome research, 15:247-256, 2007) is a report on the Fourth Elmau Conference on Nuclear Organization. This document reports that D4Z4 is an insulator element protecting from TPE. D4Z4 being able to displace a telomere from the interior of the nucleus to the periphery. This document does not describe any fragments derived from D4Z4 and does not demonstrate nor specify the insulating properties that D4Z4 might have.

It has now been found that fragments derived from the repeated D4Z4 element have insulating properties protecting from cis elements including expression regulators (enhancers, silencers), position-effect variegation and telomeric position effect. Surprisingly, even small fragments derived from a specific segment of D4Z4 exhibit these strong insulating properties.

Advantageously, the polynucleotides of this invention have the two characteristic insulating properties, in other words (i) blockage of the effect of transcription regulating elements (activating and repressing) and (ii) reduction of position-effect variegation.

Advantageously, the nucleotides of the invention also have the property of limiting the telomeric position effect.

Another advantage of this invention is that the insulating or isolating properties of the polynucleotides are not dependent on their orientation.

In a particularly advantageous manner, the polynucleotides of this invention neither positively nor negatively regulate the expression of the transgene.

The polynucleotides of the present invention are useful as insulator elements for the protection of transgene expression after transfection into eukaryote cells. These sequences can protect a transgene against the influence of chromatin environment at the site of integration. The polynucleotides according to the present invention also show an enhancer blocking activity.

### DESCRIPTION OF THE SEQUENCES

SEQ ID NO. 1: 1381 base pair polynucleotide corresponding to fragment 1-1381 of polynucleotide D4Z4.
SEQ ID NO. 2: 65 base pair polynucleotide corresponding to fragment 441-505 of polynucleotide D4Z4.
SEQ ID NO. 3: Polynucleotide D4Z4 (Genbank D38024)
SEQ ID No. 4: 432 base pair polynucleotide corresponding to fragment 382-814 of polynucleotide D4Z4.
SEQ ID No. 5: 1001 base pair polynucleotide corresponding to fragment 382-1381 of polynucleotide D4Z4.
SEQ ID No. 6: 80 base pair polynucleotide corresponding to fragment 431 to 510 of polynucleotide D4Z4.

### DESCRIPTION OF THE INVENTION

The present invention relates to polynucleotides having the sequence of SEQ ID No.1, SEQ ID No. 2, SEQ ID NO. 4, SEQ ID NO. 5 or SEQ ID NO. 6.

The invention is further related to polynucleotides with insulating properties allowing protection of the expression of a transgene from cis elements in higher eukaryotic cells selected from the group comprising the following polynucleotides:
a fragment comprising 50 to 1000 nucleotides of a polynucleotide of SEQ ID NO.1, of SEQ ID NO.2, SEQ ID NO.3, SEQ ID NO. 4, SEQ ID NO. 5 or of a polynucleotide of SEQ ID NO. 6;
a polynucleotide having at least 90% homology with a polynucleotide of SEQ ID NO. 1, of SEQ ID NO. 2, SEQ ID NO. 3, SEQ ID NO. 4, SEQ ID NO. 5 or with a polynucleotide of SEQ ID NO. 6.

Preferably, the invention concerns fragments having between 50, 65, 80, 100, 200, 400 nucleotides and 100, 500, 1000 nucleotides.

Another aspect of the present invention is an expression cassette comprising, in the direction of transcription:
a functional promoter in a host organism,
a transgene,
a terminating sequence in the same host organism,

including upstream from the promoter and/or downstream from the terminating sequence at least one polynucleotide according to the invention.

In a preferred embodiment said expression cassette comprises in the direction of transcription:
a transcription activating sequence.
a functional promoter in a host organism
a transgene,
a terminating sequence in the same host organism,

including upstream from the transcription activating sequence and/or downstream from the terminating sequence, at least one polynucleotide according to the invention.

Advantageously, said expression cassettes comprise several copies in tandem of at least one polynucleotide according to the invention.

Most preferably, said expression cassettes comprise several copies in tandem of at least one polynucleotide of SEQ ID NO. 2, of SEQ ID NO. 4 and/or of SEQ ID NO. 6.

The invention further relates to vector comprising a polynucleotide according to the invention, and/or an expression cassette according to the invention.

A further aspect of the present invention is an eukaryotic cell transformed with a polynucleotide, an expression cassette and/or a vector according to the invention.

Another aspect of the invention is a host organism comprising an expression cassette, a vector and/or a cell according to the invention. In one embodiment the host organism is non human.

The invention further relates to pharmaceutical compositions comprising an expression cassette, a vector and/or a cell according to the invention.

In a preferred embodiment, the pharmaceutical composition is for the treatment of genetic diseases, cancer, inflammatory diseases, auto-immune diseases and allergies.

In another aspect the invention relates to a method for isolating the expression of a transgene inserted into the genome of a higher eukaryotic host cell from the effect of the cis elements adjacent to the insertion site in said genome, characterized in that it includes the following steps:

a) an expression cassette and/or a vector according to the invention, comprising said transgene, is prepared,

b) the higher eukaryotic host cell is transformed with the cassette and/or vector obtained in step a).

c) the transgene is inserted into the genome of said higher eukaryotic host cell,

d) the transgene is expressed.

Preferably, the method for isolating the expression of a transgene is an in vitro method.

A further aspect of the invention is the use of a polynucleotide according to the invention in order to isolate the expression of a transgene incorporated into the genome of a higher eukaryotic host cell from the effects of cis elements.

Preferably, the invention is directed to an in vitro use.

In a preferred embodiment, the cis elements are transcription regulating elements such as activators or repressors.

In another preferred embodiment, the cis elements are topological constraints of the neighbouring chromatin regions.

The invention also relates to the use of a polynucleotide according to the invention for transgenesis, genetic transformation, gene therapy or for the production of recombinant proteins.

The invention is preferably directed to in vitro uses.

### Polynucleotides

The term “polynucleotide” according to the present invention refers to a single strand nucleotide chain or its complementary strand which can be of the DNA or RNA type, or a double strand nucleotide chain which can be of the cDNA (complementary) or genomic DNA type. Preferably, the polynucleotides of the invention are of the DNA type, namely double strand DNA. The term “polynucleotide” also refers to modified polynucleotides.

The polynucleotides of this invention are isolated or purified from their natural environment. Preferably, the polynucleotides of this invention can be prepared using conventional molecular biology techniques such as those described by Sambrook et al. (Molecular Cloning: A Laboratory Manual, 1989) or by chemical synthesis.

The polynucleotides covered by this invention are isolators or “insulators”. These polynucleotides therefore possess insulating properties which make it possible to protect the expression of a transgene from cis elements in higher eukaryotic cells. These insulating properties in higher eukaryotic cells can be tested using techniques well known to the man skilled in the art. (Gaszner & Felsenfeld. (2006). Nature Genetics Reviews. (7): 703-13). Suitable tests are also described in the examples.

The polynucleotides of the invention possess insulating properties against cis elements.

The term “insulating properties against cis elements” according to the invention refers to protection of a transgene from elements close to the insertion site in the genome. These cis elements are transcription regulators (activators repressors) and/or topological constraints on the neighbouring chromatin. When a transgene is incorporated into the genome of a host organism, the polynucleotides of the invention make it possible to isolate transcription and therefore the expression of the transgene from the cis effects of elements adjacent to the incorporation site. The polynucleotides of the invention thus protect from activating elements (enhancers) or repressor elements (silencers) of transcription. The polynucleotides of the invention isolate the expression of the transgene from cis-regulating elements by blocking these transcription regulating elements. The polynucleotides of the invention also protect the transgene from topological constraints in the neighbouring chromatin regions. They therefore protect the transgene from position-effect variegation. The polynucleotides of the invention also protect the transgene against telomeric position effect. The polynucleotides of the invention have at least one of these cis element insulating properties. Advantageously, the polynucleotides of the invention protect the transgene from both transcription regulating elements, position-effect variegation and the telomeric position effect.

In a preferred embodiment, the insulating properties of polynucleotides are not dependent on their 5′3′ orientation. The invention thus also relates complementary sequences to sequences of SEQ ID Nos. 1, 2 and 4-6.

In another preferred embodiment, the polynucleotides of the invention, neither positively nor negatively, regulate the expression of the transgene.

According to this invention, the polynucleotide of SEQ ID NO. 3 possesses cis element insulating properties but we have also shown that the polynucleotide whose sequence falls between position 1 and position 1381 of SEQ ID NO. 3 (SEQ ID NO. 1), the polynucleotide whose sequence falls between position 441 and 505 of SEQ ID NO. 3 (SEQ ID NO. 2), the polynucleotide whose sequence falls between position 382 and position 814 of SEQ ID NO. 3 (SEC) ID NO. 4), the polynucleotide whose sequence falls between position 382 and position 1381 of SEQ ID NO. 3 (SEQ ID NO. 5) and the polynucleotide whose sequence falls between position 431 and position 510 of SEQ ID NO. 3 (SEQ ID NO. 1) possess insulating properties.

The term polynucleotide “fragments” refers to a polynucleotide including part but not all of the polynucleotide from which it is derived. The fragments according to this invention retain the insulating properties of the polynucleotide from which they are derived.

The invention thus relates to a fragment of at least 50, 65, 80, 100, 500, 1000, 1500, 2000, 2500, 3000, 3300 nucleotides of the polynucleotides of SEQ ID Nos. 1-6. More preferred, the invention relates to a fragment of at least 50, 65, 80, 100, 500, 1000 nucleotides of the polynucleotides of SEQ ID Nos. 1, 2, 4-6. Preferably, the invention relates to a fragment of at least 50, 65, 80, 100, 500 nucleotides of the polynucleotides of SEQ ID Nos. 2, 4-6. Most preferably, the invention relates to a fragment of at least 50, 65, 80, 100 nucleotides of the polynucleotides of SEQ ID Nos. 2, 4 and 6.

In a preferred embodiment, the fragments according to the invention have a size comprised between 50-100, 50-200, 50-500, 50-1000, 65-100, 65-500, 65-1000, 80-100, 80-200, 80-500, 80-1000, 100-200, 100-500, 100-1000 nucleotides.

In a preferred embodiment of the invention, the fragments of the invention include the polynucleotide of SEQ ID NO. 1, 2, 4-6. In an even more advantageous embodiment of the invention, the fragments of the invention include the polynucleotide of SEQ ID NO. 2, 4 and 6.

Advantageously, the fragments according to the invention have a minimal size while retaining their insulating properties. Remarkably, the polynucleotides of SEQ ID NO. 2, SEQ ID No.4 and SEQ ID No. 6 having a length of respectively 65, 432 and 80 nucleotides possess insulating properties.

The invention also relates to polynucleotides presenting a degree of identity or homology with the polynucleotides of SEQ ID NO. 1, SEQ ID NO. 2 or SEQ ID NOs. 4-6. These polynucleotides retain the insulating properties of the reference polynucleotide.

The invention thus relates polynucleotides presenting at least 70%, 75%, 80%, 85%, 90%, 95%, 98% and preferably at least 99% identity with the polynucleotides of SEQ ID NO. 1, SEQ ID NO. 2 or SEQ ID NO. 4-6.

The term identical polynucleotides refers to nucleotides with no variation or changes between two sequences. These polynucleotides can have a deletion, addition or substitution of at least one nucleotide with respect to the reference polynucleotide.

The invention also relates to polynucleotides presenting at least 70%, 75%, 80%, 85%, 90%, 95%, 98% and preferably at least 99% homology with the polynucleotides of SEQ ID NO. 1, SEQ ID NO. 2 or SEQ ID NO. 3.

The term homology refers to the degree of resemblance between two protein or nucleic sequences. These polynucleotides can have a deletion, addition or substitution of at least one nucleotide with respect to the reference polynucleotide.

The degree of identity between two sequences, quantified by a score, is based on the percentage of identities and/or conservative substitutions in the sequences. The methods for measuring and identifying the degree of identity and degree of homology between nucleic acid sequences are well known to the man skilled in the art. For example, vectors NTi Vector NTi 9.1.0, alignment program AlignX (Clustal W algorithm) (Invitrogen INFORMAX, http://www.invitrogen.com) can be used. Preferably, the default parameters are used.

The invention also relates to polynucleotides capable of selective hybridization with polynucleotides of SEQ ID NO. 1, SEQ ID NO. 2 or SEQ ID NOs. 4-6. These polynucleotides retain the insulating properties of the reference polynucleotide.

Preferably, selective hybridization is carried out under average conditions of stringency and even more preferably under strict conditions of stringency. The term “sequence capable of selective hybridization” according to the invention refers to sequences which hybridize with the reference sequence at a significantly higher level than background noise. The level of the signal generated by interaction between the sequence capable of selective hybridization and the reference sequence is generally 10 times, preferably 100 times more intense than that for the interaction with other DNA sequences which generate background noise. The strict hybridization conditions leading to selective hybridization are well known to the man skilled in the art. In general, the hybridization and washing temperature is 5° C. below the Tm of the reference sequence at a given pH and for a given ionic force. Typically, the hybridization temperature is at least 30° C. for a 15 to 50 nucleotide polynucleotide and at least 60° C. for a polynucleotide with over 50 nucleotides. As an example, hybridization is carried out in the following buffer: 6×SSC, 50 mM Tris-HCI (pH 7.5), 1 mM EDTA, 0.02% PVP, 0.02% Ficoll, 0.02% BSA, 500 ug/ml of denatured salmon sperm DNA. Washing is carried out, for example, successively at low stringency in 2×SSC, 0.1% SDS buffer, at average stringency in 0.5×SSC, 0.1% SDS buffer and at high stringency in 0.1×SSC, 0.1% SDS. Hybridization can evidently be carried out using other methods well known to the man skilled in the art (see in particular Sambrook et al., Molecular Cloning: A Laboratory Manual, 1989). Preferably, the polynucleotides which hybridize selectively with a reference polynucleotide retain the function of the standard sequence.

### Expression Cassettes

The polynucleotides of the present invention having insulating properties are useful for a number of DNA constructs and have many different uses.

According to an embodiment of the invention, polynucleotides with insulating properties are inserted into an expression cassette using cloning techniques well known to the man skilled in the art. This expression cassette comprises the required elements for transcription and translation of the transgene of interest, including transcription initiation and termination sites.

Advantageously, this expression cassette includes elements allowing production of a polypeptide or polynucleotide by a host cell or host organism and the necessary elements for regulation of this expression.

Typically the expression cassettes include at least one functional promoter in the host organism, a transgene and a terminating sequence in the same host organism. According to the invention, the expression cassettes also include, upstream from the promoter and/or downstream from the terminating sequence, at least one polynucleotide with insulating properties according to the invention.

The term “upstream from the promoter” refers to a polynucleotide located before the promoter in the direction of transcription or in position 5′ of the promoter.

The term “downstream from the terminating sequence” refers to a polynucleotide located after the terminating sequence in the direction of transcription or in position 3′ of the terminating sequence.

Polynucleotides with insulating properties are therefore located on one and/or other side of the transgene and the necessary elements for its expression. Thus the insulating polynucleotides protect the transgene from the effect of cis elements. When the transgene is incorporated into the genome, the insulating polynucleotides also protect the neighbouring regions from the effect of the transgene and from the effect of the elements required for its expression. This can be particularly advantageous when the expression cassettes according to the invention also include transcription activating sequences.

It is understood that the insulating polynucleotides according to the invention can be located upstream from and/or downstream from the transgene and the necessary elements for its expression.

Advantageously, several copies of an insulating polynucleotide according to the invention can also be located in tandem upstream and/or downstream from the transgene. Use of several copies in tandem may make it possible to increase the insulating effect. This is particularly useful when the polynucleotide is a small-sized fragment such as the insulating polynucleotide of SEQ ID NO. 2 consisting of 65 nucleotides, the insulating polynucleotide of SEQ ID NO. 4 consisting of 432 nucleotides and the insulating polynucleotide of SEQ ID No. 6 consisting of 80 nucleotides.

Advantageously the expression cassettes according to the invention allow expression of the transgene in a higher eukaryote or mammal cell.

The term “transgene” refers to any foreign gene or any exogenous gene introduced into the organism by means of genetic modification.

A transgene is introduced into the genome of a host organism for its expression. For example, this can be a gene coding for a protein which is not usually expressed in such an organism. The transgene can be chosen from among the following: a gene used for therapy, a diagnostic gene, a gene marker such as the GFP or luciferase gene, a gene used to inhibit cell proliferation or a gene stimulating cell function. More generally, the term transgene refers to any exogenous nucleic acid molecule coding for a biological substance, either a transcript such as mRNA, rRNA, tRNA, a ribozyme or an aptazyme or a protein, polypeptide or peptide of therapeutic or experimental interest. According to the invention, the transgene includes gDNA, cDNA, natural DNA or DNA obtained totally or partially by chemical synthesis. It can also consist of an antisense sequence, mutant sequence of a given gene or a sequence involved in the transcription, expression or activation of functions of genes or even any suitable sequence for activation of pro-drugs or cytotoxic substances. The transgene of interest is placed under the control of a promoter allowing its expression in the host organism.

Any type of promoter sequence can be used in the expression cassettes according to the invention. The choice of the promoter depends on the host organism chosen for expression of the transgene. Some promoters allow constitutive expression while other promoters are, to the contrary, inducible. The promoter can also be active in certain types of cells or tissues only.

All these promoters are described in the literature and well known to the man skilled in the art. The following can be cited as examples of promoters for the expression of a transgene in higher eukaryotic cells allowing constitutive expression (CMV, SV40, Thymidine kinase, EF-1α, Ubc), inducible promoters (TeT system, inducible or repressible Ecdysone), and promoters allowing viral and lentiviral expression.

The expression cassettes according to the present invention can also include any other sequence required for expression of polypeptides or polynucleotides such as regulation elements or signal sequences allowing the secretion of polypeptides produced by the host organism. In particular, any regulation sequence leading to an increased level of expression of the transgene inserted into the expression cassette can be used. According to the invention, other regulation sequences can be used in combination with the promoter regulation sequence such as transcription activators (enhancer). The activating sequences for functional transcription in higher eukaryotic cells include in particular viral enhancers (SV40, CMV, Moloney murine leukemia virus (MMLV) LTR) and specific gene enhancers (for example, beta-globin, human beta-globin or murin, interferon responsive enhancer element).

A large variety of terminating sequences can be used in the expression cassettes according to the invention, with these sequences allowing termination of transcription and polyadenylation of mRNA. Any functional terminating sequence in the chosen host organism can be used.

### Vectors

Advantageously, the polynucleotides and expression cassettes according to this invention are inserted into a vector.

The term “vector” refers to any nucleic acid molecule capable of auto-replication into which foreign nucleic acid fragments can be introduced with the aim of incorporating them in a host cell in order to create a genetic transformation. In the broad sense, the term “vector” refers to any carrier of genetic material from one cell to another.

The techniques for construction of these vectors and of insertion of a polynucleotide of the invention into these vectors are well known to the man skilled in the art. The man skilled in the art will choose suitable vectors depending on the host organism to be transformed and as a function of the transformation technique applied. In a preferred embodiment, the invention relates to a transgenesis vector including a cassette according to the invention. Such a vector can be prepared according to methods currently used by the man skilled in the art. These vectors are intended for incorporation into the host cell. Preferably, integrative vectors are used which allow integration of the exogenous nucleic acid molecule into the genome of the host cell in a random manner. The man skilled in the art is aware of different integrative systems, plasmid or viral, chosen as a function of the host cell. Such a transgenesis vector can be used in therapy and in particular in gene therapy.

Advantageously, the vectors can include several expression cassettes separated or isolated from each other by the insulating polynucleotides of the invention.

### Transformed Eukaryotic Cells and Host Organisms

The invention also relates to eukaryotic cells transformed with a cassette according to the invention, in particular one that has incorporated such a cassette into its genome. The man skilled in the art is well aware of the standard methods for incorporation of a cassette (DNA sequence) into a host cell, for example transfection, lipofection, electroporation, microinjection, viral infection, thermal shock, transformation after chemical permeabilisation of the membrane or cell fusion. The methods of incorporating sequences into the genome of eukaryotic cells are well known to the man skilled in the art.

Any eukaryotic cells may be transformed with the polynucleotides, expression cassettes or vectors of the present invention.

In a preferred embodiment, the invention relates to eukaryotic cells from higher eukaryotes such as multicellular organisms, vertebrates and mammals.

D4Z4 has also been shown to be a binding site for CTCF (genbank accession number NC_{—}000016.8 for Homo sapiens), a DNA binding protein that is highly conserved in different species of eukaryotes. In addition, A-type Lamins (encoded by the LMNA gene, genbank accession number NC_{—}000001.9 for Homo sapiens), might contribute to D4Z4 insulator activity. Therefore the different insulator elements derived from D4Z4 are expected to work in a wide range of cells expressing these genes.

Preferably, the insulating polynucleotides of the present invention are used in eukaryotic cells expressing a CTCF protein or a homolog of a CTCF protein (Bell et al., Cell 98:387-396, 1999).

Advantageously, the invention relates to transformed cells from vertebrates. More advantageously, the invention relates to mammalian cells. Preferably the cells are animal cells transformed by the methods described in Vasquez et al. ((2001) PNAS 98, 8403-8410) and Yanez et al. ((1999), Somatic cell and molecular genetics 25, 27-31). In one embodiment of the invention, the invention relates to transformed human cells.

The term “transformation” refers to introduction of any foreign genetic material into a cell. The transformed cells typically comprise a polynucleotide, an expression cassette or a vector according to the present invention.

This invention also relates to a host organism transformed with a polynucleotide, an expression cassette or a vector according to the invention. The term host organism refers in particular, in accordance with the invention, to any multicellular organism, lower or higher. The term host organism refers to a non-human organism.

The invention also relates to an animal, with the exception of humans, containing at least one transformed cell, in other words having incorporated a polynucleotide, a cassette or a vector according to the invention.

Polynucleotides with insulating properties according to the invention make it possible to isolate a transgene in a transgenic animal. The transgene is protected from the negative or unsuitable effect of elements adjacent to the incorporation site but the insulating polynucleotides also limit the impact of experimentally incorporated sequences on endogenous elements present at the site of incorporation which can have a detrimental effect on the genetically transformed cell or organism.

The methods for obtaining transgenic animals are well known to the man skilled in the art, and are described in particular for sheep (Wilmut et al. Nature 385, 810-813, 1997; WO 97 07669), mice (Wakayama et al. Nature 394, 369-374, 1998; WO 99 37143), cattle (Wells et al. Biol. Reprod. 60, 996-1005, 1999), goats (Baguisi et al. Nature Biotechnol. 17, 456-461, 1999; WO 00 25578), pigs (Polejaeva et al. Nature 407, 86-90, 2000), rabbits (Chesne et al. Nature Biotechnol. 20, 366-369, 2002) and rats (Zhou & al., Science, 25 Sep. 2003; PCT/FR04/001275). The nuclear transfer methods are described by Campbell & al. (Nuclear transfer in practice, School of Biosciences, University of Nottingham, Leicestershire, United Kingdom).

### Pharmaceutical Compositions

According to one aspect of the invention, the expression cassettes, vectors or transformed cells according to the invention can be used for the manufacture of a drug intended for use in particular in the treatment of genetic diseases, cancer, inflammatory diseases, autoimmune diseases and allergies.

According to one aspect of the invention, the transgenesis vectors according to the invention can be used for the manufacture of a drug intended to provide treatment requiring expression of an absent or mutated gene in an organism cell. This includes in particular diseases linked to a genetic abnormality or cancer.

Transformed cells can be, according to a preferred aspect of the invention a tumour cell whose proliferation is to be controlled. In such a case, the cassette incorporated into the genome includes a transgene intended to inhibit cell proliferation.

The beta-globin insulator identified in chicken has been used in the treatment of various diseases. For example, in the case of cell therapy based on the use of adenovirus, various strategies aimed at controlling the expression and stability of the target gene have been developed. One of the first approaches was to introduce transcription terminators. Later, the use of insulating elements making it possible to block the cis-regulating sequences was found to be more effective, suggesting the importance of this type of sequence in the treatment of various pathologies. The polynucleotides, cassettes, vectors and transformed cells according to the invention can therefore also be used in gene therapy strategies or gene transfer strategies to target malignant cells and deliver toxins or treatment genes.

A detailed description of the techniques used to implement the invention can be found in classical molecular biology works such as <<Molecular cloning: a laboratory manual>>, 2nd edition, Cold Spring Harbor Laboratory Press, 1989, by Sambrook, Fritsch and Maniatis <<Short protocols in molecular biology>>, Fourth edition, by Ausubel et al.,

### FIGURES

FIG. 1: Schematic description of element D4Z4 from nucleotide 1 to 3303. The putative coding region for protein Dux4 is given in grey as are the two homeodomains it contains. The position of the 27 base pair DNA sequence (DBE) binding the protein complex YY1, HMGB2 and nucleolin is indicated (Gabellini D, Green M R, Tupler R. (2002). Cell. 110(3):339-48. WO/2005/037231).

FIG. 2: Detail of the constructions used to study the effect of D4Z4 on protection against the position effect. Various vectors containing a hygromycin resistance gene (HyTK) and an eGFP reporter gene placed under the control of CMV promoters. The vectors either carry or not a telomere seed (triangles) which allows incorporation at chromosome ends of of vectors made linear by cleavage by restriction enzymes after transfection into human cells C33A (cervical adenocarcinoma) while the other constructions are incorporated in a random manner. This incorporation is verified by in situ fluorescent hybridization on metaphasic chromosome preparations. In certain constructions, lambda phage DNA was cloned between the gene and telomer (left hand column) or after the gene in order to control that the effect of D4Z4 is not dependent on increasing the distance between the reporter gene and putative regulatory elements at the site of incorporation into the host cell. After transfection, cells which have incorporated the transgene are selected in the presence of hygromycin in a culture medium and expression of eGFP is measured as a function of time by flow cytometry.

FIG. 3: Expression of reporter gene eGFP monitored by flow cytometry for several weeks in different cell populations. Repeated element D4Z4 protects against position-effect variegation when reporter gene eGFP is incorporated in a random manner into the human genome (compare vectors GFP-D4Z4 and pCMV) and against the telomeric position effect (compare vectors GFP-D4Z4 Telo and pCMVTelo). This insulation is not dependent on increasing the distance between the reporter gene and the telomeric sequences of the host cell (compare GFP-Telo and pCMVlambda).

FIG. 4: Element D4Z4 was compared to chicken beta-globin insulator (5′ HS4, 1.2 kilobase sequence). The expression of reporter gene eGFP was measured as a function of time by flow cytometry.

FIG. 5: Vectors making it possible to test the insulating activity of a DNA sequence are given in accordance with the method of Chung J H, Whiteley M, Felsenfeld G. (1993). Cell. 74(3):505-14. When the activator (enhancer) is located in close proximity to the neomycin selection gene, K562 cells form a certain number of colonies in the presence of the drug. The number of colonies obtained acts as a standard for measurement of the insulating activity of a sequence (vector pNI, value 1 on the histogram). The number of colonies obtained when this test sequence is incorporated into the vector is established in the same way and given with respect to the number of clones for vector pNI. The results obtained for chicken beta-globin insulator are shown (FIG. (5-4)) and compared with a vector in which 2.3 kb of lambda phage DNA has been inserted between the promoter and activator (<<3-4>>).

FIG. 6: The goal of this experiment was to evaluate the capacity for D4Z4 to interfere with enhancer-promoter communication, one of the features of insulator elements. To this aim the test fragment is inserted between an enhancer a reporter gene and stably transfected into the K562 human erythroleukemia cell line. The different constructs are shown on the left. Each construct carries the neomycin resistance gene driven by the human γ-globin promoter (γ-Neo) flanked with the mouse 5′HS2 enhancer (E). Most of the constructs contain the 5′HS4 insulator upstream of the promoter in order to block the influence from regulatory elements at the site of integration allowing thereby to test only the enhancer-promoter communication. For each assay, colony number was normalized to the un-insulated control (pNI). Data are the average of three independent transfections. The mean values with S.D. are plotted. The following constructs were used: pNI, no insert; pJC3-4, 2.3 kb of λ DNA; pJC5-4, chicken β-globin 1.2 kb 5′HS4 insulator. For D4Z4, sense (pNI-D4Z4-S) and antisense (pNI-D4Z4-AS) orientation were cloned into pNI vectors between the enhancer and the reporter. D4Z4 reduced the colony number in an orientation-independent way suggesting that the repeat interferes with transcriptional enhancement. In order to distinguish insulation from repression, the right β-globin insulator protecting from the influence of regulatory elements at the site of integration was replaced by D4Z4 (p-E-D4Z4-S). In this configuration, the number of G418-resistant colonies is similar to the control indicating that D4Z4 is not a repressor. Lastly, we tested the ability of D4Z4 to enhance gene expression by removing the 5′ HS2 enhancer (E) in sense (pD4Z4-S) and antisense (pD4Z4-AS) constructs. In this assay, D4Z4 does not activate γ-Neo expression.

FIG. 7: Mapping of the regulatory fragments within D4Z4.

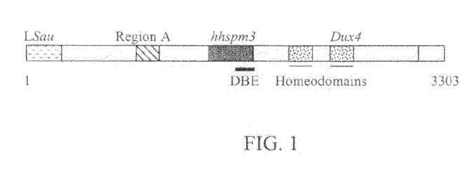

a. Schematic representation of the D4Z4 element (Genbank accession number D38024) from position 1 to 3303 given relative to the two flanking KpnI sites (K) (to scale). The different regions within D4Z4 are indicated: LSau repeat (position 1-340), Region A (position 869-1071), hhspm3 (position 1313-1780), DUX4 ORF (position 1792-3063). The different restriction sites used for the cloning of D4Z4 subfragments are indicated (B: BamHI; Bl: BlpI; F: FseI; E: EheI). b. In order to isolate the minimal fragment conferring insulator activity, different fragments obtained after digestion of D4Z4 were cloned downstream of the eGFP reporter (“C” constructs) or between the reporter and the telomeric seed (“T” constructs). Linearized plasmids were transfected into cells and the percentage of eGFP positive cells was monitored by flow cytometry for an extended period of time. The histogram represents the mean value of the percentage of eGFP positive cells from day 18 to day 29 when eGFP expression reaches a plateau±S.D shown by error bars. Fragments ΔB2-3 (position 1 to 382), ΔB1-2 (position 814 to 1381) and ΔF (position 1549 to 3303) do not abrogate TPE or PEV while fragment ΔB1 (position 1 to 1381), ΔB1-3 (position 382 to 814) and ΔE (deleted of a distal 623 by fragment from position 2269 to 2892) protect from PEV and TPE.

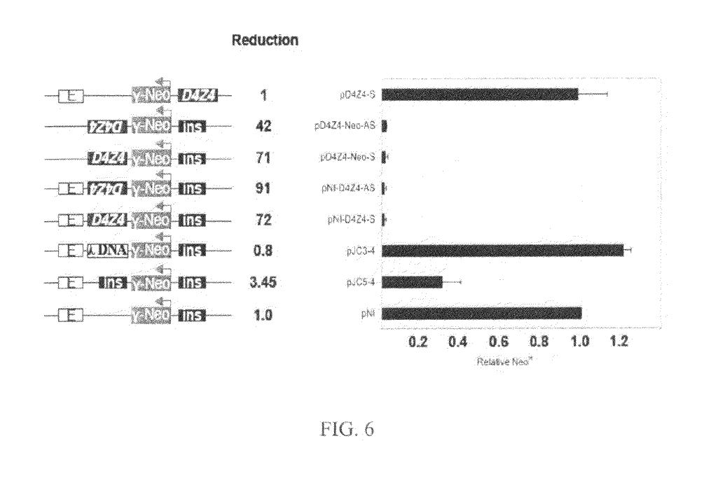

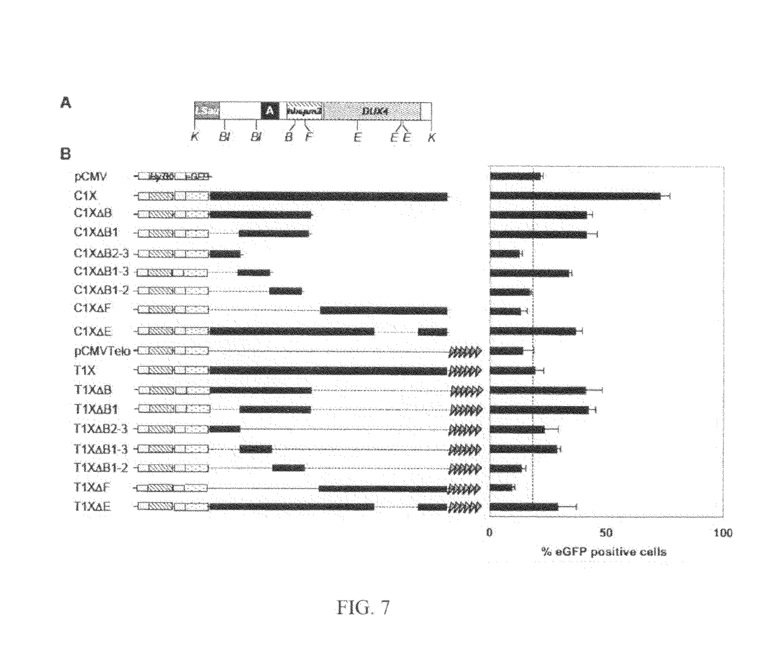

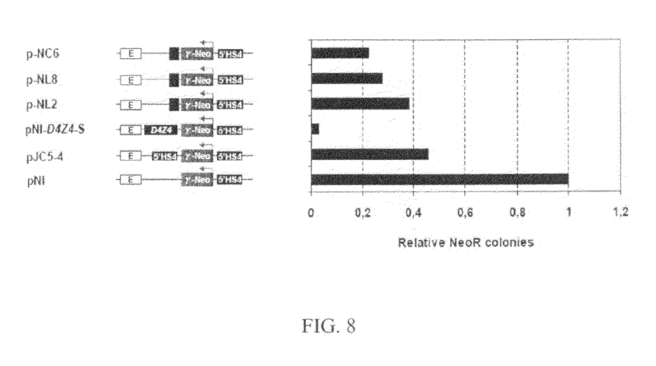

FIG. 8: In order to isolate the minimal fragment conferring enhancer blocking activity, the K562 human erythroleukemia cell line was stably transfected with the constructs shown on the left. Experiments were performed as described in FIG. 6. The following constructs were used: pNI, no insert; pJC5-4, chicken β-globin 1.2 kb 5′HS4 insulator; pNI-D4Z4-S for D4Z4 sense. In addition, short fragment of 65 by corresponding to the minimal D4Z4 insulator were cloned is sense (pNC6; pNL8) or antisense (pNL2) orientation into pNI vectors between the enhancer and the reporter. The presence of these fragments between the enhancer and the reporter reduced the colony number in an orientation-independent way suggesting that they interferes with transcriptional enhancement.

FIG. 9 : The goal of this experiment was to show the capacity of short D4Z4 subfragments to protect from the influence of cis-acting elements. Therefore, the different fragments were cloned downstream of a reporter gene whose expression was measured after transfection into human cells as described for FIG. 8A. The different constructs carry a hygromycin resistance gene fused to the herpes simplex virus type 1 thymidine kinase suicide gene (HyTK) and an eGFP reporter gene, each driven by a CMV promoter (pr). The level of eGFP was measured by flow cytometry (FACS) for an extended period of time in the presence of Hygromycin B. Histograms show the average percentage of eGFP positive cells from day 18 to day 29 of independent transfections, when eGFP expression reaches a plateau. Short fragment of 80 (Tld and Tcd) to 65 (Tda and Tcd) base pairs corresponding to D4Z4 minimal insulator were compared to one copy of the 5′ HS4 insulator (pCMV 5′ HS4) or two tandem copies of the 5′ HS4 insulator (pCMV 2×5′HS4). The presence of the minimal D4Z4 insulator fragment protects gene expression from position effect variegation.

### EXAMPLES

### Example 1

### Evaluation of Protection Against Position-Effect Variegation

We made up constructions consisting of a D4Z4 repetition cloned close to the reporter gene coding for protein eGFP (Green Fluorescent Protein) containing a selection gene for human cells (Hygromycin B) (Koering et al., 2002). The constructions were transfected into different transformed human cell lines (C33A, cervical adenocarcinoma; TE671, Rhabdomyosarcoma; K562, erythrocytic leukaemia) and immortalised murin myoblasts, C2C12. After transfection, the cells were cultured in a selective medium (hygromycin B), which only allows the growth of cells which have incorporated the transgene.

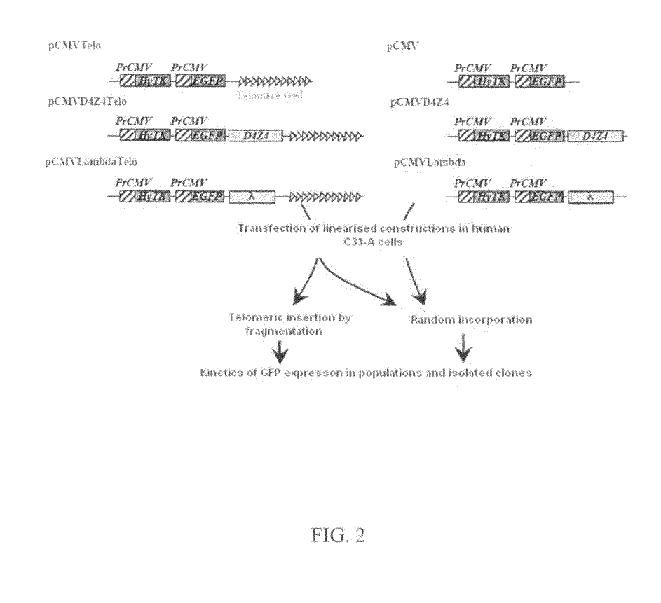

Incorporation of various transgenes was verified using cytogenetic methods (metaphasic chromosome spreads and fluorescence by in situ hybridization) and conventional molecular biology methods (Southern blot) for cellular populations or isolated clones (FIG. 2).

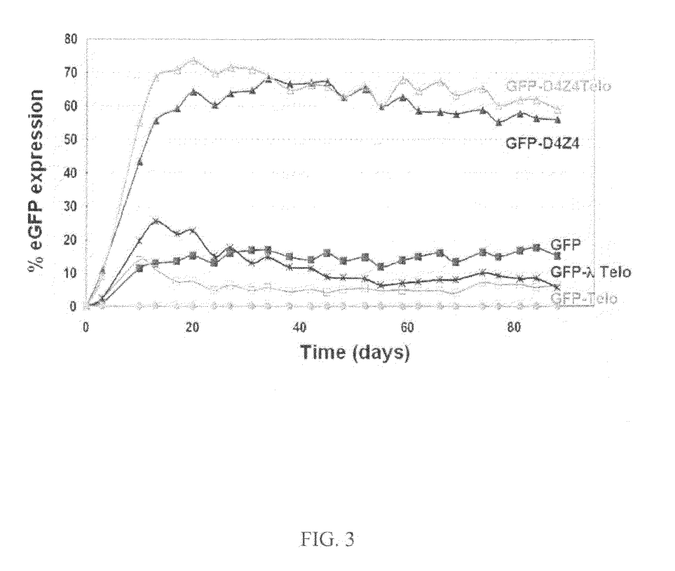

Expression of reporter gene eGFP led to the production of a fluorescent protein whose level of expression could be measured by flow cytometry at regular time intervals over several weeks (FIG. 3).

Cells which incorporated the transgene show an average level of expression dependent on constraints by the surrounding chromatin and variegation of expression in the course of time. This silencing effect was eliminated when cells were treated with a histone deacetylation inhibitor (Trichostatin A), one of the steps of heterochromatinisation. On the other hand, when element D4Z4 is present in the sequences, it makes it possible to maintain the expression of the transgene at constant level in the course of time, whatever the chromatin environment (telomere and rest of the genome).

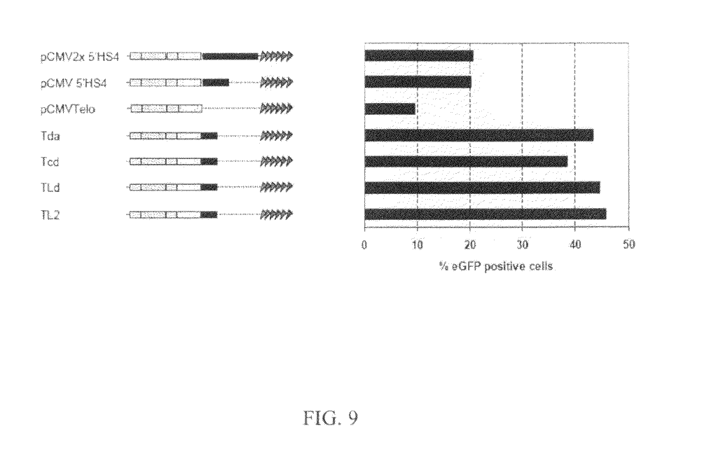

These initial results therefore demonstrate the insulating role of sequence D4Z4 of 3303 base pairs with regard to silencing by a position effect close to heterochromatin or telomeric regions. In addition, a fragment of 1381 base pairs corresponding to the 5′ end of D4Z4 from position 1 to 1381 protects expression of a reporter gene from silencing as a result of a position effect or telomeric position effect. We also isolated a short sequence of 65 base pairs (position 441 to 505 of D4Z4) and showed that this element protects expression of the reporter gene from silencing (FIG. 7).

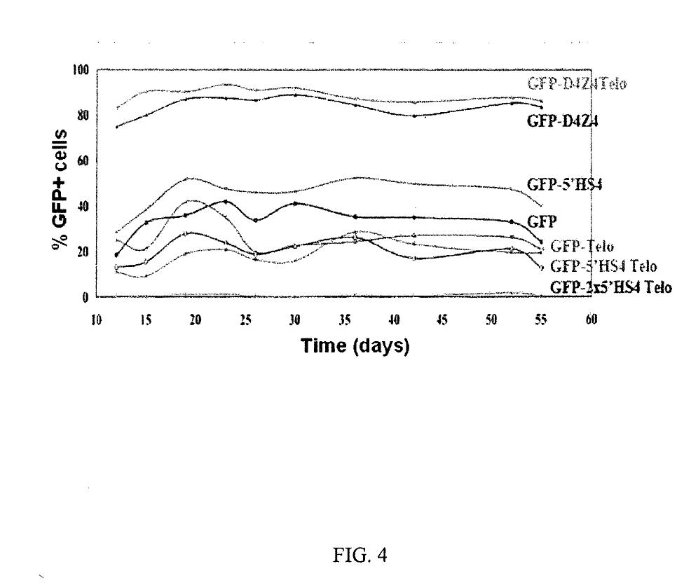

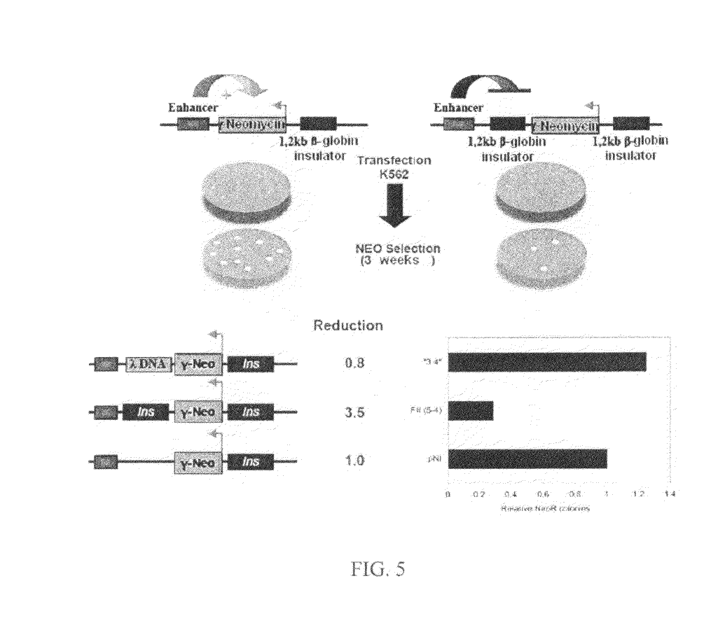

When close to a telomere, chicken beta-globin insulator incorporated in one (GFP-5′HS4) or two copies (GFP-2× 5′HS4) does not protect against the silencing effect and does not maintain a high level of the transgene in the course of time (FIG. 4). On the other hand, it protects against position-effect variegation when it is incorporated in a random manner (GFP-5′HS4) but does not lead to a high level of transgene expression.

### Example 2

### Evaluation of the Capacity to Interfere with Cis-Regulating Elements

After this, we wanted to know whether D4Z4 showed the second property characteristic of insulating elements, in other words the ability to interfere between a transcription promoter and activator or repressor by carrying out a series of different constructions.

The method we used was the same as the method employed for the description of chicken beta-globin insulator (Chung J H, Whiteley M, Felsenfeld G. (1993). Cell. 74(3):505-14).

In this type of construction, the element to be tested is incorporated between the promoter controlling expression of the neomycin resistance gene and a transcription activator. Constructions which either contain the element or not were then transfected into human cells (erythrocytic leukaemia cell line, K562) and cultured in the presence of neomycin. The number of clones which develop in the selective medium approximately 3 weeks after transfection. is directly proportional to activation of the neomycin resistance gene. Thus, if the DNA sequence inserted into the vector has the capacity to interfere between this promoter and the transcription activator than the number of colonies formed in the selective medium is significantly reduced in comparison to the controls (FIG. 5).

We used this method to evaluate the activity of D4Z4 as a (i) transcription activator (ii) repressor or (iii) insulator able to interfere between the enhancer and promoter (FIG. 6). We have shown that D4Z4 does not possess repressor or activator activity when it is located in proximity to a promoter, whatever its orientation. pN1b1X-Eα; pN1b1X-E constructions make it possible to test the activator effect. The number of clones obtained is very low compared to the control vector (pNI) which acts as a standard, thus indicating that D4Z4 does not act as a transcription activator in this context. The pNIX1X construction makes it possible to test the effect of D4Z4 on transcription repression. In this case, the number of clones obtained is similar to that obtained with the control vector (value 1).

On the other hand, it is capable of blocking the interaction between an enhancer and promoter when it is incorporated between the two, whatever its orientation (constructions pNIb1Xα and pNIb1X). The number of colonies is significantly lower with this element (91 and 71.5 times less compared to vector pNI). The beta-globin insulator (FII(5-4)) is used as control and shows a 3.45 times reduction in the number of clones compared to the control vector (pNI). When the DNA fragment of lambda bacteriophage is incorporated between the promoter controlling neomycin and the enhancer element, the number of clones which develop on the selective medium is similar to that with the pNI vector.

### REFERENCES

1. Baguisi et al. Nature Biotechnol. 17, 456-461, 1999
2. Bell A C, West A G, Felsenfeld G. (1999). Cell. 98(3):387-96
3. Chesne et al. Nature Biotechnol. 20, 366-369, 2002
4. Chung J H, Whiteley M, Felsenfeld G. (1993). Cell. 74(3):505-14
5. Gabellini D, Green M R, Tupler R. (2002). Cell. 110(3):339-48
6. Gaszner & Felsenfeld. (2006). Nature Genetics Reviews. (7): 703-13
7. Kalos M, Fournier R E. (1995). Mol Cell Biol. 15(1):198-207
8. Levelle C. and Sigal A. (Chromosome research, 15:247-256, 2007)
9. Majem M, Cascallo M, Bayo-Puxan N, Mesia R, Germa J R, Alemany R. (2006). Cancer Gene Ther. 13(7):696-705
10. Malik P, Arumugam P I, Yee J K, Puthenveetil G. (2005). Ann N Y Acad Sci. 1054:238-49
11. Ottavani et al. (Neuromuscular disorders, 16:644-726, 2006)
12. Parnell et al. (Encyclopedia of Life Sciences, 1-5, 2006)
13. Petrov et al. (PNAS, 103:18:6982-6987, 2006)
14. Polejaeva et al. Nature 407, 86-90, 2000
15. Puthenveetil G, Scholes J, Carbonell D, Qureshi N, Xia P, Zeng L, Li S, Yu Y, Hiti A L, Yee J K, Malik P. (2004). Blood. 104(12):3445-53
16. Sambrook et al. (Molecular Cloning: A Laboratory Manual, 1989)
17. Silvere et al. (Am. J. Hum. Genet., 76:375-386, 2005)
18. Tam et al. (Journal of Cell Biology, 167::2:269-279, 2004)
19. Van der Maarel S M, Frants R R. (2005). Am J Hum Genet. 76(3): 375-86
20. Van Meerten T, Claessen M J, Hagenbeek A, Ebeling S B. (2006). Gene Ther. 13(9):789-97
21. Vasquez et al. (2001) PNAS 98, 8403-8410
22. Wakayama et al. Nature 394, 369-374, 1998
23. Wells et al. Biol. Reprod. 60, 996-1005, 1999
24. Wilmut et al. Nature 385, 810-813, 1997
25. Yanez et al. (1999), Somatic cell and molecular genetics 25, 27-31
26. Zhou & al., Science, 2003

1-15. (canceled)

16. Polynucleotide selected from the group consisting of:
a) a polynucleotide comprising the sequence of SEQ ID NO:1, SEQ ID NO:2, SEQ ID NO:4, SEQ ID NO:5, or SEQ ID NO:6;
b) a fragment comprising 50 to 1000 nucleotides of a polynucleotide of SEQ ID NO:1, SEQ ID NO:2, SEQ ID NO:4, SEQ ID NO:5 or of a polynucleotide of SEQ ID NO:6 and having insulating properties allowing protection of the expression of a transgene from cis elements in higher eukaryotic cells; and
c) a polynucleotide having at least 90% homology with a polynucleotide of SEQ ID NO:1, of SEQ ID NO:2, SEQ ID NO:4, SEQ ID NO:5, or with a polynucleotide of SEQ ID NO:6 and having insulating properties allowing protection of the expression of a transgene from cis elements in higher eukaryotic cells.

17. Expression cassette comprising, in the direction of transcription, a functional promoter in a host organism, a transgene and a terminating sequence in the same host organism, wherein said expression cassette includes upstream from the promoter and/or downstream from the terminating sequence at least one polynucleotide selected from:
a) a polynucleotide comprising the sequence of SEQ ID NO:1, SEQ ID NO:2, SEQ ID NO:4, SEQ ID NO:5, or SEQ ID NO:6;
b) a fragment comprising 50 to 1000 nucleotides of a polynucleotide of SEQ ID NO:1, SEQ ID NO:2, SEQ ID NO:4, SEQ ID NO:5 or of a polynucleotide of SEQ ID NO:6, and having insulating properties allowing protection of the expression of a transgene from cis elements in higher eukaryotic cells; and
c) a polynucleotide having at least 90% homology with a polynucleotide of SEQ ID NO:1, SEQ ID NO:2, SEQ ID NO:4, SEQ ID NO:5, or with a polynucleotide of SEQ ID NO:6, and having insulating properties allowing protection of the expression of a transgene from cis elements in higher eukaryotic cells.

18. Expression cassette according to claim 17 comprising in the direction of transcription, a transcription activating sequence, a functional promoter in a host organism, a transgene and a terminating sequence in the same host organism, wherein said expression cassette includes upstream from the transcription activating sequence and/or downstream from the terminating sequence, at least one polynucleotide selected from:
a) a polynucleotide comprising the sequence of SEQ ID NO:1, SEQ ID NO:2, SEQ ID NO:4, SEQ ID NO:5, or SEQ ID NO:6;
b) a fragment comprising 50 to 1000 nucleotides of a polynucleotide of SEQ ID NO:1, SEQ ID NO:2, SEQ ID NO:4, SEQ ID NO:5, or of a polynucleotide of SEQ ID NO:6, and having insulating properties allowing protection of the expression of a transgene from cis elements in higher eukaryotic cells;
c) a polynucleotide having at least 90% homology with a polynucleotide of SEQ ID NO:1, SEQ ID NO:2, SEQ ID NO:4, SEQ ID NO:5, or with a polynucleotide of SEQ ID NO:6, and having insulating properties allowing protection of the expression of a transgene from cis elements in higher eukaryotic cells.

19. Expression cassette according to claim 17 comprising several copies in tandem of said polynucleotide.

20. Expression cassette according to claim 18 comprising several copies in tandem of said polynucleotide.

21. Vector comprising a polynucleotide according to claim 1.

22. Vector comprising an expression cassette according to claim 2.

23. Eukaryotic cell comprising an expression cassette according to claim 17.

24. Host organism comprising an expression cassette according to claim 17.

25. Pharmaceutical composition comprising an expression cassette according to claim 17.

26. Pharmaceutical composition comprising a eukaryotic cell according to claim 23.

27. Method for isolating the expression of a transgene inserted into the genome of a higher eukaryotic host cell from the effect of the cis elements adjacent to the insertion site in said genome, comprising:
a) preparing an expression cassette comprising, in the direction of transcription, a functional promoter in a host organism, a transgene and a terminating sequence in the same host organism, wherein said expression cassette includes upstream from the promoter and/or downstream from the terminating sequence at least one polynucleotide selected from:
i) a polynucleotide comprising the sequence of SEQ ID NO:1, SEQ ID NO:2, SEQ ID NO:4, SEQ ID NO:5, or SEQ ID NO:6;
ii) a fragment comprising 50 to 1000 nucleotides of a polynucleotide of SEQ ID NO:1, SEQ ID NO:2, SEQ ID NO:4, SEQ ID NO:5, or of a polynucleotide of SEQ ID NO:6, and having insulating properties allowing protection of the expression of a transgene from cis elements in higher eukaryotic cells; and
iii) a polynucleotide having at least 90% homology with a polynucleotide of SEQ ID NO:1, of SEQ ID NO:2, SEQ ID NO:4, SEQ ID NO:5, or with a polynucleotide of SEQ ID NO:6, and having insulating properties allowing protection of the expression of a transgene from cis elements in higher eukaryotic cells;
b) transforming the higher eukaryotic host cell with the expression cassette of a);
c) inserting the expression cassette comprising the transgene into the genome of said higher eukaryotic host cell; and
d) expressing the transgene.

28. Method according to claim 27 wherein the cis elements are transcription regulating elements.

29. Method according to claim 28 wherein the transcription regulating elements are activators or repressors.

30. Method according to claim 27 wherein the cis elements are topological constraints of the neighbouring chromatin regions.

31. Method for isolating the expression of a transgene inserted into the genome of a higher eukaryotic host cell from the effect of the cis elements adjacent to the insertion site in said genome, comprising:
a) preparing an expression cassette comprising, in the direction of transcription, a transcription activating sequence, a functional promoter in a host organism, a transgene and a terminating sequence in the same host organism, wherein said expression cassette includes upstream from the transcription activating sequence and/or downstream from the terminating sequence, at least one polynucleotide selected from:
i) a polynucleotide comprising the sequence of SEQ ID NO:1, SEQ ID NO:2, SEQ ID NO:4, SEQ ID NO:5, or SEQ ID NO:6;
ii) a fragment comprising 50 to 1000 nucleotides of a polynucleotide of SEQ ID NO:1, of SEQ ID NO:2, SEQ ID NO:4, SEQ ID NO:5, or of a polynucleotide of SEQ ID NO:6, and having insulating properties allowing protection of the expression of a transgene from cis elements in higher eukaryotic cells; and
iii) a polynucleotide having at least 90% homology with a polynucleotide of SEQ ID NO:1, of SEQ ID NO:2, SEQ ID NO:4, SEQ ID NO:5, or with a polynucleotide of SEQ ID NO:6, and having insulating properties allowing protection of the expression of a transgene from cis elements in higher eukaryotic cells;
b) transforming the higher eukaryotic host cell with the expression cassette of a);
c) inserting the expression cassette comprising the transgene into the genome of said higher eukaryotic host cell; and
d) expressing the transgene.

32. Method according to claim 31 wherein the cis elements are transcription regulating elements.

33. Method according to claim 32 wherein the transcription regulating elements are activators or repressors.

34. Method according to claim 31 wherein the cis elements are topological constraints of the neighbouring chromatin regions.


SEQUENCE LISTING (6 sequences)

SEQ ID NO: 1 - DNA, 1381 bp, Homo sapiens
ggtaccagcaggtgggccgcctactgcgcacgcgcgggtttgcgggcagccgcctgggctgtgggagcagcccgggccagagctctcctgcctctccaccagcccaccccgccgcctgaccgccccctccccaccccccaccccccacccccggaaaacgcgtcgtcccctgggctgggtggagacccccgtcccgcgaaacaccgggccccgcgcagcgtccgggcctgacaccgctccgccggctcgcctcctcctgtcgcccccgggccaccgtcgcccgcccgcccgggcccctgcgggcccctgcagccgcccagctgccagcacgggcggctggcggcggaacgcagaccccaggcccggcgcacaccggggacgctgagcgttccaggcgggagggaaggcgggcagagatggagagaggaacgggagagactagaggggcggaagggacgttaggagggaggcagggaggcagggaggcagggaggaacggagggagagacagagcgacgcagggactgggggcggggccgagggagccggggacggacggggggaggaaggcagggaggaaaagcggtcctcggcctccgggagtagcgggaccgcccgcctccgggaaaacggtcagcgtccggcgcgggctgagggctgggcccacagccgccgcgccggccggcggggcaccacccattcgccccggttccggggcccagggagtgggcggtttcctccgggacaaaagaccgggactcgggttgccgtcgggtcttcacccgcgcggttcacagaccgcacatccccaggctgagccctgcaacgcggcgcgaggcccacagacccggccacggaggagccacacgcaggacgacggaggcgtgattttggtttccgagtggctttgccctcccgaaggcggcctgttgctcacgtctctccggcccccgaaaggctggccatgccgactgtttgctcccggagctctgcggcacccggaaacatgcagggaagggtgcaagcccggcatggtgccttcgctctccttgccaggttccaaacccgccacactgcagactcccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgg

SEQ ID NO: 2 - DNA, 65 bp, Homo sapiens
agaggggcggaagggacgttaggagggaggcagggaggcagggaggcagggaggaacggagggag

SEQ ID NO: 3 - DNA, 3303 bp, Homo sapiens
ggtaccagcaggtgggccgcctactgcgcacgcgcgggtttgcgggcagccgcctgggctgtgggagcagcccgggccagagctctcctgcctctccaccagcccaccccgccgcctgaccgccccctccccaccccccaccccccacccccggaaaacgcgtcgtcccctgggctgggtggagacccccgtcccgcgaaacaccgggccccgcgcagcgtccgggcctgacaccgctccgccggctcgcctcctcctgtcgcccccgggccaccgtcgcccgcccgcccgggcccctgcgggcccctgcagccgcccagctgccagcacgggcggctggcggcggaacgcagaccccaggcccggcgcacaccggggacgctgagcgttccaggcgggagggaaggcgggcagagatggagagaggaacgggagagactagaggggcggaagggacgttaggagggaggcagggaggcagggaggcagggaggaacggagggagagacagagcgacgcagggactgggggcggggccgagggagccggggacggacggggggaggaaggcagggaggaaaagcggtcctcggcctccgggagtagcgggaccgcccgcctccgggaaaacggtcagcgtccggcgcgggctgagggctgggcccacagccgccgcgccggccggcggggcaccacccattcgccccggttccggggcccagggagtgggcggtttcctccgggacaaaagaccgggactcgggttgccgtcgggtcttcacccgcgcggttcacagaccgcacatccccaggctgagccctgcaacgcggcgcgaggcccacagacccggccacggaggagccacacgcaggacgacggaggcgtgattttggtttccgagtggctttgccctcccgaaggcggcctgttgctcacgtctctccggcccccgaaaggctggccatgccgactgtttgctcccggagctctgcggcacccggaaacatgcagggaagggtgcaagcccggcatggtgccttcgctctccttgccaggttccaaacccgccacactgcagactcccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctcccccttcctcttcgtctctccggccccaccaccaccaccgccaccacgcctcccccaccaccccccccccccaccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctggctggctggctgtccgggcaggcctcctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggccacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagagggggtctcccaacctgccccggcgcgcggggatttcgcctacgccgccccggctcctccggagccggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcaggccaaggggtgcttgcgccacccacgtcccaggggagtccgtggtggggctggggccggggtccccaggtcgccggggcggcgtgggaaccccaagccggggcagctccacctccccagcccgcgcccccgggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggcgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtccaggcccggtgagagactccacaccgcggagaactgccattctttcctgggcatcccggggatcccagagccggccca

SEQ ID NO: 4 - DNA, 432 bp, Homo sapiens
tgagcgttccaggcgggagggaaggcgggcagagatggagagaggaacgggagagactagaggggcggaagggacgttaggagggaggcagggaggcagggaggcagggaggaacggagggagagacagagcgacgcagggactgggggcggggccgagggagccggggacggacggggggaggaaggcagggaggaaaagcggtcctcggcctccgggagtagcgggaccgcccgcctccgggaaaacggtcagcgtccggcgcgggctgagggctgggcccacagccgccgcgccggccggcggggcaccacccattcgccccggttccggggcccagggagtgggcggtttcctccgggacaaaagaccgggactcgggttgccgtcgggtcttcacccgcgcggttcacagaccgcacatccccaggc

SEQ ID NO: 5 - DNA, 1001 bp, Homo sapiens
gctgagcgttccaggcgggagggaaggcgggcagagatggagagaggaacgggagagactagaggggcggaagggacgttaggagggaggcagggaggcagggaggcagggaggaacggagggagagacagagcgacgcagggactgggggcggggccgagggagccggggacggacggggggaggaaggcagggaggaaaagcggtcctcggcctccgggagtagcgggaccgcccgcctccgggaaaacggtcagcgtccggcgcgggctgagggctgggcccacagccgccgcgccggccggcggggcaccacccattcgccccggttccggggcccagggagtgggcggtttcctccgggacaaaagaccgggactcgggttgccgtcgggtcttcacccgcgcggttcacagaccgcacatccccaggctgagccctgcaacgcggcgcgaggcccacagacccggccacggaggagccacacgcaggacgacggaggcgtgattttggtttccgagtggctttgccctcccgaaggcggcctgttgctcacgtctctccggcccccgaaaggctggccatgccgactgtttgctcccggagctctgcggcacccggaaacatgcagggaagggtgcaagcccggcatggtgccttcgctctccttgccaggttccaaacccgccacactgcagactcccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgg

SEQ ID NO: 6 - DNA, 80 bp, Homo sapiens
gggagagactagaggggcggaagggacgttaggagggaggcagggaggcagggaggcagggaggaacggagggagagaca